(19) United States
(12) Patent Application Publication
(10) Pub. No.: US 2022/0289000 A1
(43) Pub. Date: Sep. 15, 2022

(54) AIR SCENTING DEVICE FOR INTRODUCING AT LEAST ONE FRAGRANCE INTO THE VEHICLE INTERIOR OF A MOTOR VEHICLE

(71) Applicant: Mahle International GmbH, Stuttgart (DE)

(72) Inventors: Daniel Eberle, Ludwigsburg (DE); Simon Dangelmaier, Fellbach (DE); Sham Kasar, Stuttgart (DE); Sven Petzner, Welzheim (DE); Oliver Schultze, Stuttgart (DE)

(21) Appl. No.: 17/689,979

(22) Filed: Mar. 8, 2022

(30) Foreign Application Priority Data
Mar. 9, 2021 (DE) 102021202292.8

(51) Int. Cl. B60H 3/00; A61L 9/01

(52) U.S. Cl. CPC B60H 3/0035; B60H 3/0028; A61L 9/01; A61L 2209/133

(57) ABSTRACT

An air scenting device for introducing at least one fragrance into a vehicle interior of a motor vehicle is disclosed. The air scenting device includes at least one fragrance cartridge arranged in a housing interior of a housing. An adjustment mechanism is provided for adjusting at least one flap closure between an open position, where the flap closure releases at least one of an inlet opening and an outlet opening of a cartridge housing, and a closed position, where the flap closure closes the at least one of the inlet opening and the outlet opening. A fragrance inlet is disposed on the housing for introducing air to be admixed with fragrance, and a fragrance outlet is disposed on the housing for discharging the air admixed with fragrance.

### CROSS-REFERENCE TO RELATED APPLICATION

This application claims priority to German Application No. DE 10 2021 202 292.8 filed on Mar. 9, 2021, the contents of which are hereby incorporated by reference in its entirety.

### TECHNICAL FIELD

The invention relates to an air scenting device for introducing at least one fragrance into the vehicle interior of a motor vehicle.

### BACKGROUND

So-called air scenting devices are used in modern motor vehicles to admix the air present in the vehicle interior with a fragrance. Such a fragrance is typically perceived as pleasant by the vehicle occupants and thus increases the driving comfort.

So-called fragrance cartridges are typically used here, which are filled with a fragrance of a certain composition and concentration. An air inlet and an air outlet are typically provided on the cartridge housing of such a fragrance cartridge, so that air can be guided through the interior of the cartridge housing and admixed with the fragrance at the same time. The air exiting from the cartridge thus contains the desired fragrance.

It has proven to be disadvantageous in such conventional concepts that only a single fragrance of a certain composition and concentration is present in the fragrance cartridge, which limits the variability in admixing the air with this fragrance. In particular, it is only possible with difficulty or not in all in this way to vary the concentration of fragrance and the composition of the fragrance specifically to the application and situation.

### SUMMARY

It is therefore the object of the present invention to disclose new ways in the development of air scenting devices, in particular for scenting air in vehicle interiors.

This object is achieved by the subject matter of the independent claim(s). Preferred embodiments are the subject matter of the dependent claims.

The basic concept of the invention is accordingly to design an air scenting device so that it is capable of accommodating two or more fragrance cartridges, which each contain a fragrance of individual composition and/or concentration. The air scenting device is designed according to the invention so that, by means of a shared adjustment mechanism, individual fragrance cartridges can be individually released for air to flow through and thus to admix this air with fragrance. In this way, it is possible to admix air with a fragrance of a certain composition and certain concentration specifically to the application and situation.

Such an air scenting device according to the invention comprises a housing, which delimits a housing interior through which air can flow. The air scenting device additionally comprises at least one fragrance cartridge arranged in the housing interior, which has a cartridge housing in which a fragrance can be accommodated or is accommodated. In the simplest case, the air scenting device can thus operate as a device having only one certain fragrance. However, two or more such fragrance cartridges are typically arranged in the housing interior to be able to implement the advantage according to the invention of the present invention, namely providing fragrance in a flexible manner, in particular specifically to the application and situation.

The cartridge housing of the at least one fragrance cartridge has an inlet opening for introducing the air into the cartridge housing, so that the air can be admixed with the fragrance present therein as it flows through the cartridge housing. The cartridge housing furthermore has an outlet opening for discharging the air admixed with the fragrance from the cartridge housing. The air scenting device furthermore has at least one adjustable flap closure for alternately opening or closing inlet opening and outlet opening. Such a flap closure is expediently provided for each fragrance cartridge present in the housing interior. Furthermore, the air scenting device comprises an adjustment mechanism for adjusting the at least one flap closure between an open position and a closed position. In the open position, the flap closure releases the inlet and outlet openings of the assigned fragrance cartridge, so that air can flow into the relevant cartridge housing to be admixed with fragrance and subsequently can flow back out of this cartridge housing. In the closed position, the flap closure closes the inlet and outlet openings in a fluid-tight manner, so that air cannot flow through these two openings. In this way, it is possible to guide the air to be admixed with fragrance deliberately through individual fragrance cartridges and to admix it in these fragrance cartridges with the fragrance present therein.

Furthermore, the air scenting device comprises a fragrance inlet formed on the housing to introduce the air to be admixed with fragrance into the housing interior and a fragrance outlet formed on the housing for discharging air admixed with fragrance from the at least one fragrance cartridge out of the housing interior.

According to one preferred embodiment, the housing is formed to be cup-like and has a housing base, in which the fragrance outlet is preferably arranged, and from which, preferably vertically, a circumferential wall of the housing forming a cup collar protrudes, in which the fragrance inlet is preferably arranged. An air scenting device designed in this way has a particularly compact structure and can be installed in a space-saving manner in a motor vehicle in this way.

The cup-like housing is preferably formed open and has a cup opening, which is closable or closed by means of a housing cover of the housing. This enables simple installation and removal of individual fragrance cartridges in the housing interior of the air scenting device.

The cup-like housing preferably has an essentially cylindrical geometrical shape, so that the at least one fragrance cartridge is insertable through the cup opening into the housing interior and is preferably detachably fastenable there on the housing base. This variant also facilitates the insertion and removal of individual fragrance cartridges in the housing of the air scenting device.

According to one advantageous refinement, at least two fragrance cartridges are arranged detachably fastenable or detachably fastened on the housing base of the housing. The detachable fastening enables a simple replacement of individual fragrance cartridges, in particular when their store of fragrance is exhausted. In addition, it can be individually established which fragrance cartridges having which fragrance composition or concentration are to be provided and used.

According to a further advantageous refinement, the housing comprises a partition wall, which divides the housing interior into an air distributor space communicating fluidically with the inlet opening of the at least one fragrance cartridge and an air collector space communicating fluidically with the outlet opening of the at least one fragrance cartridge. The division is implemented in such a way that the air distributor space only communicates fluidically with the air collector space via the at least one fragrance cartridge, but not directly. In this way, controlled guiding of the air through the housing interior and controlled permeation of the air with a certain fragrance is possible. In particular, this prevents air introduced into the housing interior from being guided past all fragrance cartridges present in the housing interior in the manner of a bypass, without being admixed with fragrance, if this is not explicitly desired—by closing all flap closures.

According to one preferred embodiment, the air distributor space communicates fluidically with the respective inlet opening of all fragrance cartridges arranged in the housing interior. In this embodiment, the air collector space communicates fluidically with the respective outlet opening of all fragrance cartridges arranged in the housing interior. In this way, it is possible to supply the air introduced into the housing interior and to be admixed with fragrance individually to the desired fragrance cartridge and to discharge it from the housing interior again after it flows through this fragrance cartridge, in which the air was admixed with fragrance. A complex air duct structure for distributing and collecting the air can therefore be omitted in this embodiment, which is accompanied by not insignificant cost advantages.

A flap closure adjustable by means of the adjustment mechanism is particularly preferably provided for each fragrance cartridge provided in the housing interior. In this way, it can be individually established into which of the provided fragrance cartridges the air to be admixed with fragrance is to be introduced. In this way, it can be individually established which fragrance—i.e., having which concentration or composition—is to be added to the air guided through the housing interior.

According to one preferred embodiment, the at least one flap closure is designed in the manner of an adjustable flip valve having a valve flap for closing inlet opening and outlet opening, wherein the valve flap is arranged in a rotationally-fixed manner on a valve shaft of the flip valve, which is designed to be rotationally adjustable relative to the housing. In this variant, the flip valve has a guide element interacting with an adjustable link guide, so that the flip valve is adjustable between its open position and its closed position by adjusting the link guide of the at least one flap closure. This embodiment enables the existing flap closures or flip valves to be adjusted with the aid of only a single link guide.

All provided flap closures are particularly preferably designed as flip valves having a respective guide element. Each guide element interacts with the link guide so that all flip valves can be adjusted by means of the link guide. This embodiment thus enables the provided flap closures or flip valves to be adjusted with the aid of only a single link guide. Said link guide can be driven here by means of a suitable, in particular electromechanical actuator.

The adjustment mechanism or the link guide can particularly expediently be designed and adapted to the at least two flap closures so that at most one of the at least two flap closures can always be located in its open position. The link guide can be designed to be rotationally adjustable in relation to the housing, in particular rotatable around an axial axis or center longitudinal axis of the housing. The link guide expediently has a symmetrical contour for this purpose, which is provided with an asymmetrical section, which deviates from the symmetrical contour to break symmetry. That flap closure, the guide element of which is in the region of the asymmetrical section, will be or is adjusted into the open position, whereas the other flap closures, the guide elements of which are arranged outside the asymmetrical section in the link guide, are located in their closed position. In this way, a single flip valve or flap closure can be adjusted from the closed position into the open position and back into the closed position again, whereas all other flip valves or flap closures remain in the closed state here.

The air scenting device particularly preferably comprises a purge valve adjustable between an open position and a closed position, via which purging air can be introduced into the air collector space and can flow there to the fragrance outlet. In this way, the air collector space can be purged of residues of fragrance possibly present therein.

According to a further preferred embodiment, the purge valve can also be adjusted by means of the adjustment mechanism between its open position and its closed position. The interaction of the adjustment mechanism with the purge valve is particularly expediently implemented, as also in the case of the other flap closures or flip valves, so that the purge valve is only in the open state when all flap closures or flip valves which are assigned to the fragrance cartridges are in the closed state and are closed. In this way, it is ensured that the purging air introduced by means of the purge valve into the air collector space cannot enter the fragrance cartridges possibly provided therein and mix there in an undesired manner with the fragrance provided in the fragrance cartridge.

The invention furthermore relates to a motor vehicle having a vehicle body and a vehicle interior, in which at least one air scenting device according to the invention as presented above is arranged. The above-explained advantages of the air scenting device are therefore also transferred to the motor vehicle according to the invention.

Further important features and advantages result from the dependent claims, the drawings, and the associated description of the figures with reference to the drawings.

It is obvious that the features mentioned above and to be explained hereinafter are usable not only in the respective specified combination, but also in other combinations or alone, without leaving the scope of the present invention.

Preferred exemplary embodiments of the invention are shown in the drawings and are explained in more detail in the following description, wherein identical reference signs refer to identical or similar or functionally identical components.

### BRIEF DESCRIPTION OF THE DRAWINGS

In the figures, which are each schematic:

FIG. 1 shows an example of an air scenting device according to the invention in a perspective illustration,

FIG. 2 shows the air scenting device of FIG. 1 in a sectional illustration,

FIG. 3 shows the air scenting device of FIGS. 1 and 2 in an axial top view of an upper side of the housing facing toward the cup opening,

FIG. 4 shows the air scenting device of FIGS. 1 and 2 in an axial top view of a lower side opposite to the upper side,

FIG. 5 shows the air scenting device in a top view of the support structure in the axial direction,

FIG. 6 shows the illustration of FIG. 4 without fragrance cartridges.

### DETAILED DESCRIPTION

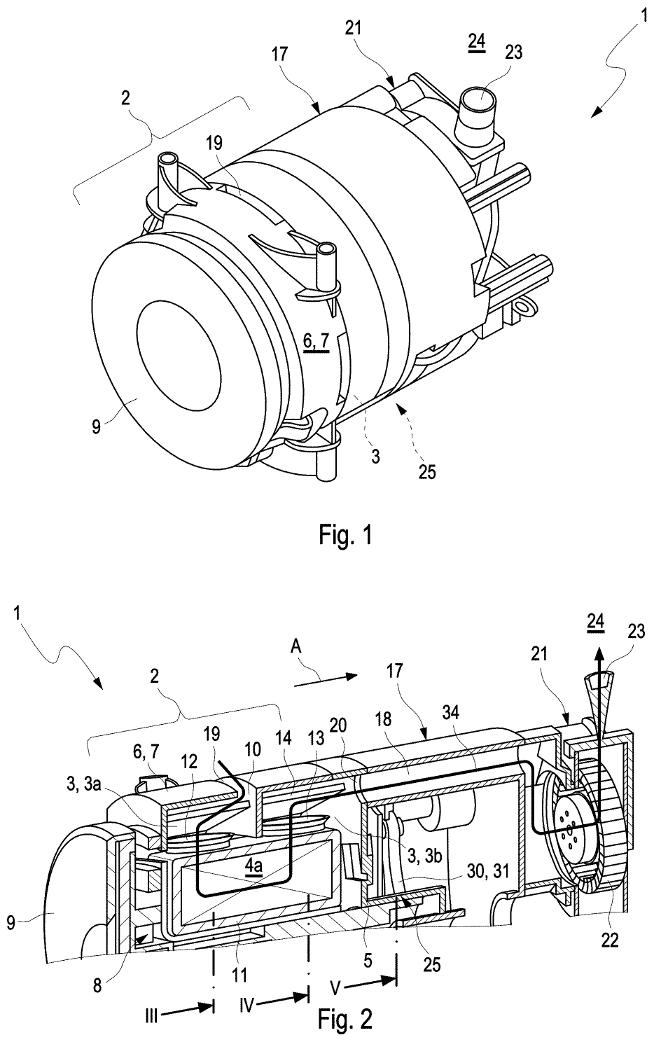

FIG. 1 illustrates an example of an air scenting device 1 according to the invention in a perspective illustration. FIG. 2 shows the air scenting device 1 of FIG. 1 in a sectional illustration.

As may be seen from FIG. 1, the air scenting device 1 comprises a housing 2, which delimits a housing interior 3 through which air can flow. Four fragrance cartridges 4a-4d are arranged in the housing interior 3, of which only a single fragrance cartridge 4a is shown in FIG. 2. According to FIGS. 1 and 2, the housing 2 is formed cup-like and has a housing base 5, from which a circumferential wall 7 of the housing 2 forming a cup collar 6 protrudes vertically. A fragrance inlet 19 for introducing air to be admixed with fragrance into the housing interior 3 is present in the cup collar 6 or in the circumferential wall 7. Correspondingly, a fragrance outlet 20 for discharging the air admixed with fragrance from the housing interior 3 is provided in the housing base 5.

As may be seen from FIGS. 1 and 2, the cup-like housing 2 has an essentially cylindrical geometrical shape. The housing 2 is formed open in an axial direction A and has a cup opening 8, which can be closed by means of a housing cover 9 of the housing 2. In this way, the fragrance cartridges 4a-4d can be introduced through the cup opening 9 into the housing interior 3 and detachably fastened there on the housing base. To facilitate the installation, suitable positioning aids 15 having axially extending positioning rails 16 can be provided in the housing interior 3, by means of which the fragrance cartridges 4a-4d can be brought into the provided fastening position.

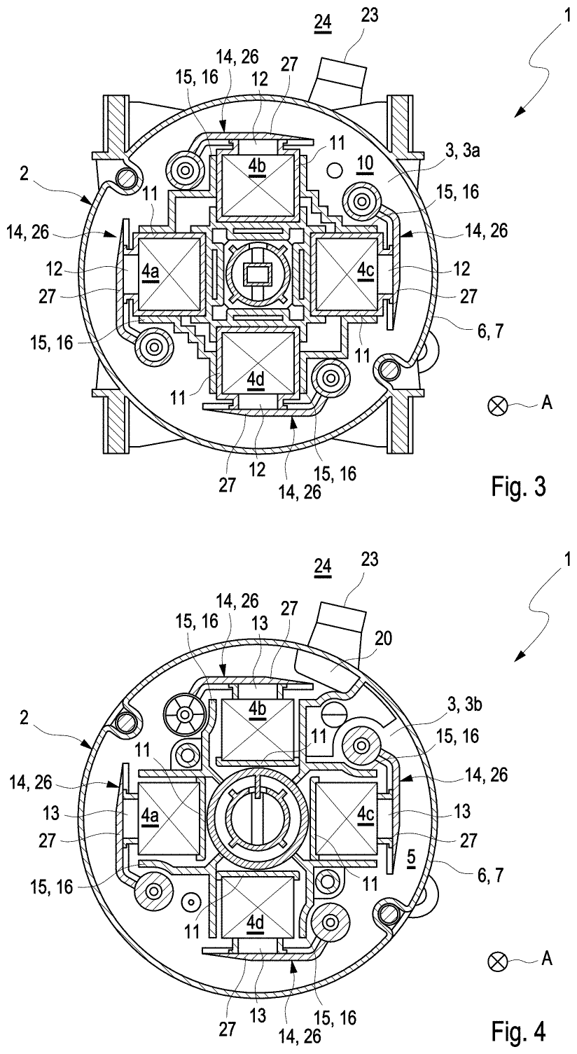

FIG. 3 shows the air scenting device 1 of FIGS. 1 and 2 in a top view of an upper side 5a of the housing base 5 of the housing 2 facing toward the cup opening 8 in the axial direction A. FIG. 3 illustrates that four fragrance cartridges 4a-4d are arranged on the housing base 5 in the housing interior 3. The fragrance cartridges 4a-4d are detachably fastened on the housing base 5 independently of one another by means of a respective clip or catch connection (not shown).

FIG. 4 shows the air scenting device 1 of FIGS. 1 and 2 in a top view of a lower side 5b of the housing base 5 of the housing 2 opposite to the upper side 5a opposite to the axial direction A.

It may also be inferred from FIGS. 3 and 4 together with the illustration of FIG. 2 that each of the four fragrance cartridges 4a-4d comprises a cartridge housing 11 in which a fragrance (not shown) is provided. Each cartridge housing 11 has an inlet opening 12 for introducing the air into the cartridge housing 11. The fragrance provided in the cartridge housing 11 can thus mix with the air introduced into the cartridge housing 11, i.e., the air is admixed in the cartridge housing 11 with the respective fragrance provided therein. After this mixing process, the air can be discharged from the cartridge housing 11 again via an outlet opening 13 also provided in the cartridge housing 11.

It is obvious that an individual fragrance can be provided in each fragrance cartridge 4a4d, thus in each cartridge housing 11. However, it is also conceivable to provide the same fragrance, but in different concentration, in two or more of the fragrance cartridges 4a-4d.

It may furthermore be inferred from FIGS. 2 and 3 that each of the four fragrance cartridges 4a-4d has an adjustable flap closure 14, by means of which the respective inlet opening 12 and outlet opening 13 of the assigned fragrance cartridge 4a-4d can alternately be opened or closed.

As may be seen from FIGS. 2 and 3, the housing 2 comprises a partition wall 10 arranged in the housing interior 3. This divides the housing interior 3 into an air distributor space 3a communicating fluidically with the inlet openings 12 of the fragrance cartridges 4a-4d and an air collector space communicating fluidically with the outlet openings 12 of the fragrance cartridges 4a-4d. The partition wall 10 is designed so that the air distributor space 3a only communicates fluidically with the air collector space 3b via the fragrance cartridges 4a-4d, but not directly. This means that no air can be guided past the fragrance cartridges 4a-4d having the respective fragrance in terms of a bypass. The air distributor space 3a additionally communicates fluidically with the respective inlet opening 13 of all fragrance cartridges 4a-4d arranged in the housing interior 3. The air collector space 14 accordingly communicates fluidically with the respective outlet opening 14 of all fragrance cartridges 4a-4d arranged in the housing interior 3. The flow path of the arrow through the housing interior 3 is identified in FIG. 2 by an arrow having the reference numeral 34.

As FIGS. 1 and 2 illustrate, a support structure 17 for the housing 2 adjoins the housing 2 axially opposite to the housing cover 9, in which structure an air duct 18 is provided, which communicates at one end fluidically with the fragrance outlet 20 and at the other end with a fan 21, which adjoins the support structure 17 and has a fan wheel 22. In this way, the air admixed with fragrance can be suctioned out of the housing interior 3 by means of the fan 21 and discharged via an outlet nozzle 23 arranged downstream of the fan wheel 22 to the external surroundings 24 of the air scenting device 1.

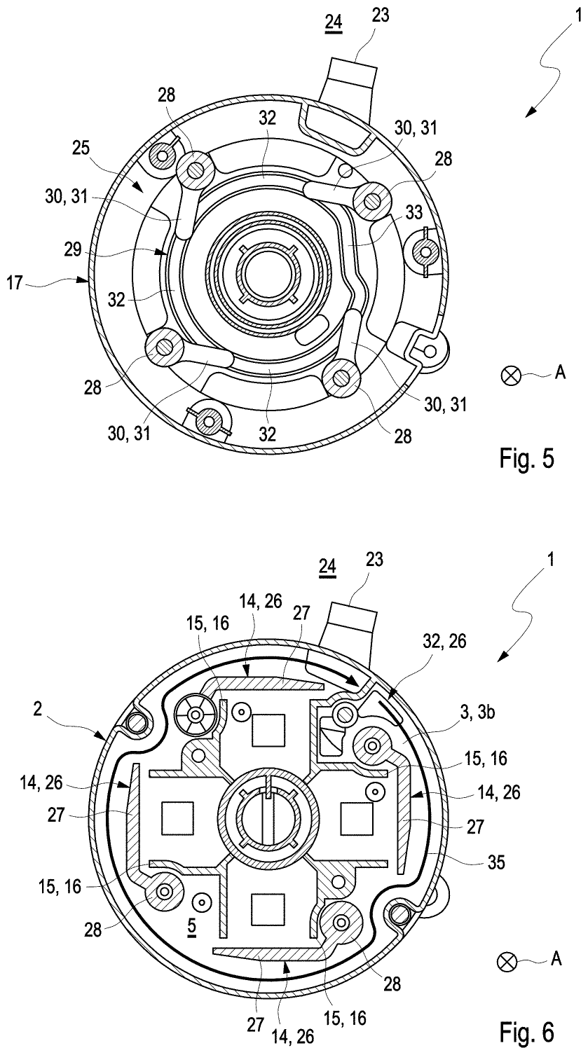

FIG. 5 shows the air scenting device 1 in a top view of the support structure 17 against the axial direction A. The illustration of FIG. 5 illustrates an adjustment mechanism 25, which forms a part of the air scenting device 1 and is used to adjust the flap closures 14. The flap closures 14 can each be adjusted by means of the adjustment mechanism 25 between an open position, in which the respective flap closure 14 releases the inlet and outlet opening 12, 13, so that air can flow into the relevant cartridge housing 11 to be admixed with fragrance and can flow back out of it, and a closed position, in which the respective flap closure 14 closes both the inlet opening 13 and also the outlet opening 14 fluid-tight, so that air and also fragrance cannot flow through these two openings 13, 14.

Each of the flap closures 14 can be designed as shown in the example of the figures in the manner of an adjustable flip valve 26 having a valve flap 27 for closing inlet opening 12 and outlet opening 13, wherein the valve flap 27 is arranged in a rotationally-fixed manner on an axially extending valve shaft 28. The valve shaft 28 is in turn designed to be rotatable relative to the housing 2. The valve shaft 28 can be rotatably mounted, for example, on the housing 2 or on the support structure.

As FIGS. 1 and 2 show, an adjustable link guide 29 rotatable in a plane perpendicular to the axial direction A, which forms a part of the adjustment mechanism 24, can be arranged axially between the housing 2 and the support structure 17.

According to FIG. 5, a guide element 30 designed in the manner of an extension 31 is provided on each valve shaft 28, which interacts with the link guide 29. These guide elements 30 are also part of the adjustment mechanism 25. The interaction takes place in such a way that successively each one of the flap closures 14 is adjusted from its closed position into the open position and back into its closed position by a rotational movement of the link guide 29. The interaction or the adjustment is thus implemented so that at an arbitrary point in time, at most one of the flap closures 14 is in its open position. In other words, it is ensured that two or more flap closures 14 are not adjusted simultaneously into the open position at any point in time. To functionally implement the adjustment mechanism 25 in this way, the link guide 29 can have, for example, a fundamentally symmetrical contour—in the example of FIG. 5 a circular contour 32 having an asymmetrical section 33, which deviates from the symmetrical, circular formation. All provided flap closures 14 are thus each formed as a flip valve 26 having a respective guide element 30 and each guide element 30 interacts with the shared link guide 29 so that the flip valves 26 can be adjusted by means of the link guide 29.

The illustration of FIG. 6 corresponds to the illustration of FIG. 4, thus shows the air scenting device 1 of FIGS. 1 and 2 in a top view of the lower side 5b of the housing base 5 against the axial direction A. In contrast to FIG. 4, the fragrance cartridges 11 are omitted in the drawing in FIG. 6.

According to FIG. 6, the air scenting device 1 can comprise a purge valve 32 adjustable between an open position and a closed position, via which purging air can be introduced into the air collector space 3b and can flow there to the fragrance outlet 20. The purge valve 33 can be designed similarly to the flap closures 14 as a flip valve 26, so that the above explanations in this regard also apply mutatis mutandis to the purge valve 33. The adjustment of the purge valve 32 can thus also take place by means of the shared adjustment mechanism 25 (cf. FIG. 5). The air path of the purging air through the air collector space 3b is identified in FIG. 6 by an arrow having the reference numeral 33. The purge valve 33 can be pre-tensioned toward the closed position by means of a pre-tensioning element, such as a spring-elastic component, for example, a coiled spring.

1. An air scenting device for introducing at least one fragrance into a vehicle interior of a motor vehicle, comprising:
a housing that delimits a housing interior through which air can flow;
at least one fragrance cartridge arranged in the housing interior, the at least one fragrance cartridge including a cartridge housing that accommodates a fragrance, wherein the cartridge housing has an inlet opening for introducing the air into the cartridge housing, so that it can be admixed with the fragrance provided therein as it flows through the cartridge housing, and wherein the cartridge housing has an outlet opening for discharging the air admixed with the fragrance from the cartridge housing,
at least one adjustable flap closure for alternately opening or closing at least one of the inlet opening and the outlet opening,
an adjustment mechanism for adjusting the at least one flap closure between an open position where the at least one flap closure releases the at least one of the inlet opening and the outlet opening, so that air can flow into the relevant cartridge housing to be admixed with fragrance and can flow back out of it again, and a closed position where the at least one flap closure closes the at least one of the inlet opening and the outlet opening in a fluid-tight manner, so that air cannot flow through the at least one of the inlet opening and the outlet opening,
a fragrance inlet disposed on the housing for introducing air to be admixed with fragrance into the housing interior and having a fragrance outlet disposed on the housing for discharging air admixed with fragrance from the at least one fragrance cartridge out of the housing interior.

2. The air scenting device as claimed in claim 1, wherein the housing is structured cup-like and has a housing base, in which the fragrance outlet is arranged, and from which a circumferential wall of the housing forming a cup collar protrudes, in which the fragrance inlet is arranged.

3. The air scenting device as claimed in claim 2, wherein the cup-like housing is structured open and has a cup opening, which is closable via a housing cover of the housing.

4. The air scenting device as claimed in claim 2, wherein the cup-like housing has a cylindrical geometrical shape, such that the at least one fragrance cartridge is insertable through the cup opening into the housing interior and is fastenable on the housing base.

5. The air scenting device as claimed in claim 1, wherein the at least one fragrance cartridge includes at least two fragrance cartridges arranged detachably fastenable on the housing base of the housing in the housing interior.

6. The air scenting device as claimed in claim 1, wherein the housing includes a partition wall that divides the housing interior into an air distributor space communicating fluidically with the inlet opening of the at least one fragrance cartridge and an air collector space communicating fluidically with the outlet opening of the at least one fragrance cartridge, such that the air distributor space only communicates fluidically with the air collector space via the at least one fragrance cartridge, but not directly.

7. The air scenting device as claimed in claim 6, wherein:
the air distributor space communicates fluidically with the inlet opening of the at least one fragrance cartridges arranged in the housing interior; and
the air collector space communicates fluidically with the outlet opening of the at least one fragrance cartridge arranged in the housing interior.

8. The air scenting device as claimed in claim 1, further comprising a flap closure adjustable via the adjustment mechanism provided for the at least one fragrance cartridge provided in the housing interior.

9. The air scenting device as claimed in claim 1, wherein:
the at least one flap closure is an adjustable flip valve having a valve flap for closing the at least one of the inlet opening and the outlet opening, wherein the valve flap is arranged in a rotationally-fixed manner on a valve shaft of the flip valve, which is rotationally adjustable relative to the housing, and
the flip valve has a guide element interacting with an adjustable link guide such that by adjusting the link guide, the flip valve is adjustable between the open position and the closed position.

10. The air scenting device as claimed in claim 9, wherein all provided flap closures are designed as a flip valve having a respective guide element and each guide element interacts with the link guide so that the flip valve can be adjusted by means of the link guide.

11. The air scenting device as claimed in claim 1, wherein the adjustment mechanism is designed and adapted to at least two flap closures so that at most one of the at least two flap closures can always be located in the open position.

12. The air scenting device as claimed in claim 1, further comprising a purge valve adjustable between an open position and a closed position, via which purging air can be introduced into the air collector space and can flow there to the fragrance outlet.

13. The air scenting device as claimed in claim 12, wherein the purge valve is adjustable via the adjustment mechanism between the open position and the closed position.

14. A motor vehicle, comprising:
a vehicle body,
at least one air scenting device arranged in a vehicle interior of the vehicle body, at least one air scenting device including:
a housing that delimits a housing interior through which air can flow;
at least one fragrance cartridge arranged in the housing interior, the at least one fragrance cartridge including a cartridge housing that accommodates a fragrance, wherein the cartridge housing has an inlet opening for introducing the air into the cartridge housing, so that it can be admixed with the fragrance provided therein as it flows through the cartridge housing, and wherein the cartridge housing has an outlet opening for discharging the air admixed with the fragrance from the cartridge housing,
at least one adjustable flap closure for alternately opening or closing at least one of the inlet opening and the outlet opening,
an adjustment mechanism for adjusting the at least one flap closure between an open position where the at least one flap closure releases the at least one of the inlet opening and the outlet opening, so that air can flow into the relevant cartridge housing to be admixed with fragrance and can flow back out of it again, and a closed position where the at least one flap closure closes the at least one of the inlet opening and the outlet opening in a fluid-tight manner, so that air cannot flow through the at least one of the inlet opening and the outlet opening,
a fragrance inlet disposed on the housing for introducing air to be admixed with fragrance into the housing interior and having a fragrance outlet disposed on the housing for discharging air admixed with fragrance from the at least one fragrance cartridge out of the housing interior.

15. The motor vehicle according to claim 14, wherein the housing is structured cup-like and has a housing base, in which the fragrance outlet is arranged, and from which a circumferential wall of the housing forming a cup collar protrudes, in which the fragrance inlet is arranged.

16. The motor vehicle according to claim 15, wherein the cup-like housing is structured open and has a cup opening, which is closable via a housing cover of the housing.

17. The motor vehicle according to claim 15, wherein the cup-like housing has a cylindrical geometrical shape, such that the at least one fragrance cartridge is insertable through the cup opening into the housing interior and is fastenable on the housing base.

18. The motor vehicle according to claim 14, wherein the at least one fragrance cartridge includes at least two fragrance cartridges arranged detachably fastenable on the housing base of the housing in the housing interior.

19. The motor vehicle according to claim 14, further comprising a flap closure adjustable via the adjustment mechanism provided for the at least one fragrance cartridge provided in the housing interior.

20. The motor vehicle according to claim 14, further comprising a purge valve adjustable between an open position and a closed position, via which purging air can be introduced into the air collector space and can flow there to the fragrance outlet.
